(19) United States
(12) Patent Application Publication
(10) Pub. No.: US 2008/0167603 A1
(43) Pub. Date: Jul. 10, 2008

(54) Method and device to prevent ventilator acquired pneumonia using nitric oxide

(76) Inventors: Alex Stenzler, Long Beach, CA (US); Arthur Samuel Slutsky, Toronto (CA)

Correspondence Address: Sidley Austin LLP, 555 West 5th Street, Suite 4000, Los Angeles, CA, 91723, US

(21) Appl. No.: 11/981,570

(22) Filed: Oct. 30, 2007

Related U.S. Application Data
(63) Continuation of application No. 11/978,940, filed on Oct. 29, 2007.
Application No. 11/978,940 is a continuation-in-part of application No. 10/944,479, filed on Sep. 17, 2004.
Application No. 10/944,479 is a continuation of application No. 10/172,270, filed on Jun. 14, 2002, now Pat. No. 6,793,644.
Application No. 10/172,270 is a continuation of application No. 09/749,022, filed on Dec. 26, 2000, now Pat. No. 6,432,077.

(51) Int. Cl. A61M 37/00; A61M 16/00

(52) U.S. Cl. 604/26; 128/204.18

(57) ABSTRACT

A respiratory assist device and method for the prevention of ventilator acquired pneumonia in a patient is described. The respiratory assist device administers nitric oxide to the oropharyngeal area in order to decontaminate or prevent the contamination of secretions that collect in the oropharyngeal area during intubation of the patient. The respiratory assist device and method may be adapted for use, for example, as an endotracheal tube or as a tracheotomy tube.

### PRIORITY CLAIM

This application is a continuation of U.S. application Ser. No. (to be assigned), “Method and Device to Prevent Ventilator Acquired Pneumonia Using Nitric Oxide,” filed Oct. 29, 2007 under Attorney Docket No. 23086-82701.

This application is also a continuation-in-part of U.S. application Ser. No. 10/944,479, filed Sep. 17, 2004. U.S. application Ser. No. 10/944,479 is a continuation of U.S. application Ser. No. 10/172,270, filed Jun. 14, 2002 and issued as U.S. Pat. No. 6,793,644, which in turn is a continuation of U.S. application Ser. No. 09/749,022, filed on Dec. 26, 2000 and issued as U.S. Pat. No. 6,432,077.

### FIELD OF THE INVENTION

The field of the invention relates to devices and methods for preventing ventilator acquired pneumonia in intubated mammals, and more specifically in mechanically ventilated human patients.

### BACKGROUND OF THE INVENTION

Ventilator acquired pneumonia (VAP) is an iatrogenic complication associated with some patients who require mechanical ventilation for more than a few days. A major causative mechanism is bacterial contamination of the lung by micro-aspiration of secretions in the upper airway that accumulate above the balloon cuff of an endotracheal or tracheotomy tube. The endotracheal or tracheotomy tube is used to deliver gas from a mechanical ventilator to the patient's lungs and the balloon cuff inflates to seal the lungs from the outside so that the pressure from the ventilator can be kept in the lungs. If there is any leak around the cuff, the contaminated secretions can seep into the lungs and cause VAP. VAP is a major cause of in-hospital mortality and morbidity for ventilated patients.

Aspiration of the subglottic secretions has been shown to reduce the incidence of early VAP in intubated, mechanically ventilated patients. Rello, J., et al., Pneumonia in intubated patients: role of respiratory airway care, Am. J. Respir. Crit. Care Med. 154:111 (1996); Valles, J., et al., Continuous aspiration of subglottic secretions in preventing ventilator associated pneumonia, Ann Intern. Med. 122:179 (1995). However, if aspiration is incomplete, there is a risk that secretions can still enter the lungs and cause VAP. Moreover, aspiration does not kill the microorganism and these microorganisms can still contaminate additional secretions or unremoved materials.

To decontaminate the secretions, others have proposed the use of silver-coated endotracheal tubes. Hartmann, M., et al., Reduction of the bacterial load by the silver-coated endotracheal tube (SCET), a laboratory investigation, Technol. Health Care. 7(5):359-70 (1999). The inflated cuff of the endotracheal tube, however, centers the tube in the trachea and typically causes secretions to pool at the sides of the inflated cuff away from the tube. Accordingly, much if not all of the contaminated secretions do not contact the tube and are not decontaminated.

Nitric oxide has been previously shown to have anti-microbial properties and has been proposed for treatment of respiratory infections. PCT/CA99/01123, published Jun. 2, 2000; Webert, K., et al., Effects of inhaled nitric oxide in a rat model of Pseudomonas aeruginosa pneumonia, Crit. Care Med. 28(7):2397-2405 (2000). However, due to the potential for toxicity of nitric oxide in the lungs, either because of its conversion to nitrogen dioxide or the formation of methomoglobin in the blood, higher concentrations of nitric oxide for inhalation has been avoided.

All of the patents and references above are incorporated by reference herein, and the description herein of problems and disadvantages of known apparatus, methods, and devices is not intended to limit the invention to the exclusion of these known entities. Indeed, embodiments of the invention may include one or more of the known apparatus, methods, and devices without suffering from the disadvantages and problems noted herein.

### SUMMARY OF THE INVENTION

Nitric oxide can be used to decontaminate the oropharyngeal area of an intubated mammal such as a mechanically ventilated human patient and to prevent ventilator acquired pneumonia, while minimizing the risk of nitric oxide gas inhalation.

In one aspect of the invention, nitric oxide is delivered to the oropharyngeal area of an intubated mammal to decontaminate the oropharyngeal area and kill or inhibit the growth of microorganisms that may grow in this area. The decontamination of the oropharyngeal area lead to the prevention of VAP. Preferably, nitric oxide gas is delivered to the oropharyngeal area at higher concentrations ranging from about 100 ppm to about 20,000 ppm.

In another aspect of the invention, a respiratory assist device is provided for use to deliver nitric oxide gas to the oropharyngeal area of an intubated mammal and may be used, for example, as an endotracheal tube or tracheotomy tube. Preferably, an inflated balloon cuff at about the distal end of the respiratory assist device acts to substantially seal the mammal's lungs from atmospheric air and also prevents nitric oxide gas that is delivered to the oropharyngeal area from entering the lungs. The respiratory assist device preferably includes tubing and portholes or exit openings for delivering nitric oxide gas to the oropharyngeal area just above the inflated balloon cuff.

The above aspects of the invention are advantageous because higher concentrations of nitric oxide gas can be used while minimizing the risk of toxicity associated with inhaling high concentrations of nitric oxide gas.

### BRIEF DESCRIPTION OF THE DRAWINGS

FIG. 1 illustrates a respiratory assist device that delivers exogenous nitric oxide (NO) gas to a location between a balloon cuff and the proximal end of the device.

FIG. 2 illustrates the respiratory assist device with a nitric oxide gas source used as an endotracheal tube to treat an intubated human patient.

FIG. 3 illustrates a respiratory assist device that delivers exogenous nitric oxide (NO) gas to a location between a balloon cuff and the proximal end of the device and aspirates secretions.

FIG. 4 depicts a S. aureus dosage curve for exposure to gaseous NO (gNO) with bacteria grown on solid media. Relative percentages of the growth of S. aureus colony forming units (cfu) at 50, 80, 120 and 160 parts per million (ppm) of nitric oxide compared with growth of S. aureus cfu in medical air (100%) are shown.

FIG. 5 depicts a Pseudomonas aeruginosa dosage curve for exposure to gNO with bacteria grown on solid media. Relative percentages of the growth of P. aeruginosa colony forming units (cfu) at 50, 80, 120 and 160 parts per million (ppm) of nitric oxide compared with growth of P. aeruginosa cfu in medical air (100%) are shown.

FIG. 6 depicts the bacteriocidal effect of 200 ppm gNO on a variety of microbes.

### DETAILED DESCRIPTION OF THE PREFERRED EMBODIMENTS

As used throughout this disclosure, the singular forms “a,” “an,” and “the” include plural reference unless the context clearly dictates otherwise. All technical and scientific terms used herein have the same meanings as commonly understood by one of ordinary skill in the art to which this invention belongs, excepting terms, phrases, and other language defined herein. All publications mentioned herein are cited for the purpose of describing and disclosing the embodiments. Nothing herein is to be construed as an admission that the embodiments described are not entitled to antedate such disclosures by virtue of prior invention.

Before the present devices and processes are described, it is to be understood that this invention is not limited to the particular devices, processes, methodologies or protocols described, as these may vary. It is also to be understood that the terminology used in the description is for the purpose of describing the particular versions or embodiments only, and is not intended to limit the scope of the present invention which will be limited only by the appended claims. For simplicity, each reference referred to herein shall be deemed expressly incorporated by reference in its entirety as if fully set forth herein.

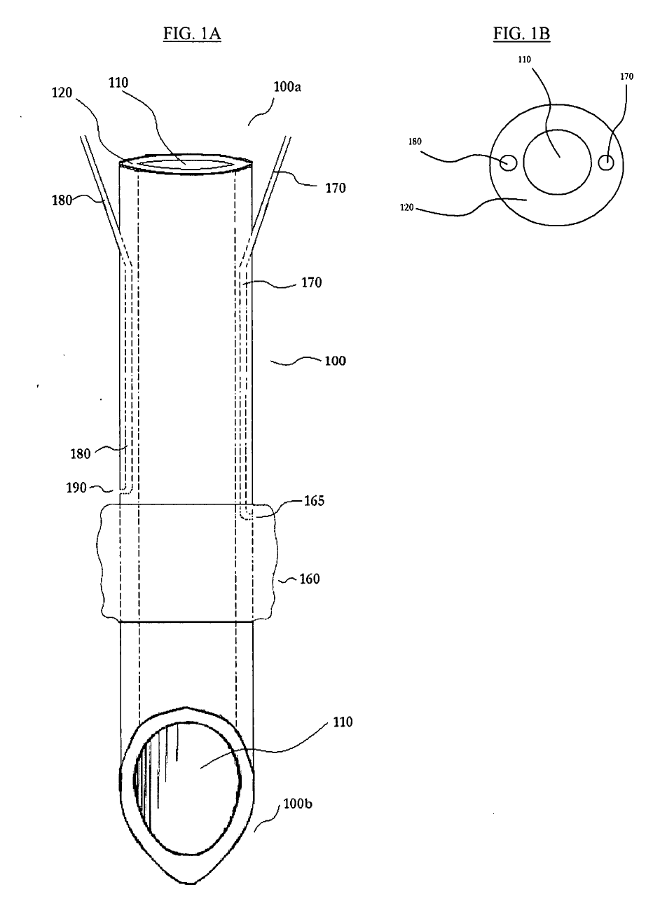

Preferred embodiments now will be described in conjunction with the figures. FIG. 1, embodiment A, illustrates an exemplary respiratory assist device 100 with a proximal end 100a and a distal end 100b and embodiment B illustrates a cross-section of the device. The respiratory assist device comprises a catheter 120 defining a central lumen 110 that receives breathable gas (such as medical oxygen and medical or atmospheric air) from a breathable gas source and delivers the breathable gas to the lungs. The respiratory assist device may also include external markings showing distances (for example in millimeters, centimeters, inches, and so forth) from its distal end to aid in its insertion into the trachea. The breathable gas source may also be used in conjunction with mechanical ventilation or other devices that aid in the ventilation of the lungs and respiration of the patient.

Close to the distal end 100b of the respiratory assist device 100 is an inflatable balloon cuff 160. An inflation air tube 170 feeds any suitable type of gas (such as air) into the balloon cuff to inflate the cuff and provide a seal within the trachea. Preferably, the balloon cuff is inflated to a pressure of about 20-30 cm H₂O, but the pressure may vary depending on the patient and size of the individual. In any event, the goal is to inflate the cuff pressure until a minimal cuff leak is noted without impeding bloodflow and without inducing tracheal stenosis. By sealing off the trachea from the lungs with the inflated balloon cuff, a deadspace cavity is formed in the oropharyngeal area in which nitric oxide gas can be topically delivered to the cavity and cavity walls with minimal entry of the NO gas into the lungs. Nitric oxide gas then may kill or inhibit the growth of microorganisms such as bacteria, fungi, or viruses that may grow in this area.

Preferably, NO gas is delivered by the respiratory assist device 100 above the proximal end of the balloon cuff 160 through the exit opening 190 that is in fluid communication with the NO gas tube 180. Thus, the nitric oxide gas flows from a nitric oxide gas source through the NO gas tube 180 and exits into the oropharyngeal area via the exit opening 190.

In the example illustrated in FIG. 1, embodiments A and B, the inflation air tube 170 is integrated into the wall of the catheter 120 of the respiratory assist device 100 and is in fluid communication with the balloon cuff 160 via an opening 165 in the wall of the catheter that leads into the interior of the cuff. Additionally, in the illustrated example the NO gas tube 180 is integrated into the wall of the catheter 120 of the respiratory assist device 100 and is in fluid communication with the oropharyngeal area via an exit opening 190. However, it will be appreciated that other configurations of the inflation air tube 170 and NO gas tube 180 also can be utilized.

For example, the respiratory assist device can have more than one inflation air tube and more than one NO gas tube. Additionally, each inflation air tube and NO gas tube can branch into multiple openings at its terminus in order to more effectively distribute inflation air to the balloon cuff or NO gas to the oropharyngeal area, respectively. Furthermore, each inflation air tube and NO gas tube, instead of being integrated into the catheter 120 wall, alternatively can be disposed either on the external surface of the respiratory assist device 100 or on the interior surface of the central lumen 110 defined by the catheter of the device. If the inflation air tube and/or NO gas tube are disposed on the interior or exterior of the catheter wall, a suitable adhesive or other means can be used to attach the inflation air tube and NO gas tube to the catheter.

In the case of the NO gas tube 180, it is preferred to provide the exit openings 190 close to the proximal end of the balloon cuff in order to directly bathe the balloon cuff or bubble through the secretions that may accumulate on the balloon cuff. However, the position of the exit openings 190 also may be located elsewhere. For example, the exit openings 190 through which NO gas is distributed by the respiratory assist device also may be positioned both at the proximal end of the balloon cuff and along the longitudinal length of the catheter such that the entire oropharyngeal area can be bathed directly with nitric oxide gas.

Regarding the nitric oxide gas source, various ways also can be used to provide this source. Preferably, the nitric oxide gas is provided from a source, such as a tank, that is pre-mixed to the desired concentration of nitric oxide so that no further dilution of the gas is necessary. For example, a common source of nitric oxide gas in hospitals is a pressurized cylinder that contains gaseous nitric oxide. The cylinder includes pressure regulators and valves for controlling the flow of nitric oxide from the cylinder into a delivery line. Alternatively, the nitric oxide gas can be diluted with a diluent gas, preferably an inert gas such as N₂ in order to minimize the breakdown of nitric oxide gas into nitrogen dioxide. Other diluent gases such as air or oxygen also can be used in order to prevent the growth of anaerobic microorganisms in the oropharyngeal area. However, diluent gases that do not react with nitric oxide gas to produce other nitrogen oxides such as nitrogen dioxide are preferred. Also, the nitric oxide gas and diluent gas preferably are mixed either actively using a gas blender or passively using a tee-connection. The concentration of the nitric oxide gas can be controlled by controlling the amount of dilution. Examples of nitric oxide delivery systems that can be used to deliver nitric oxide gas are described in U.S. Pat. Nos. 6,432,077 and 6,5812,599, issued to one of the applicants, and are hereby incorporated by reference as if fully set forth herein.

Nitric oxide gas can also be provided from nitric oxide releasing compounds such as potassium nitrate, nitroglycerin, diphenyl nitrosamine, and ammonium compounds. Nitric oxide releasing compounds can be provided in a device having a chamber in which released nitric oxide gas can be channeled and stored. Examples of such a container is described in PCT/CA/99/01123 published on Jun. 2, 2000, which is hereby incorporated by reference. Various other means of providing nitric oxide gas also can be used including producing nitric oxide from air by using electricity as described in U.S. Pat. No. 5,396,882, which is hereby incorporated by reference.

Preferably, the concentrations of nitric oxide and nitrogen dioxide are also monitored using NO/NOx sensors that are commercially available, for example, from Pulmonox Medical Incorporated (Alberta, Canada).

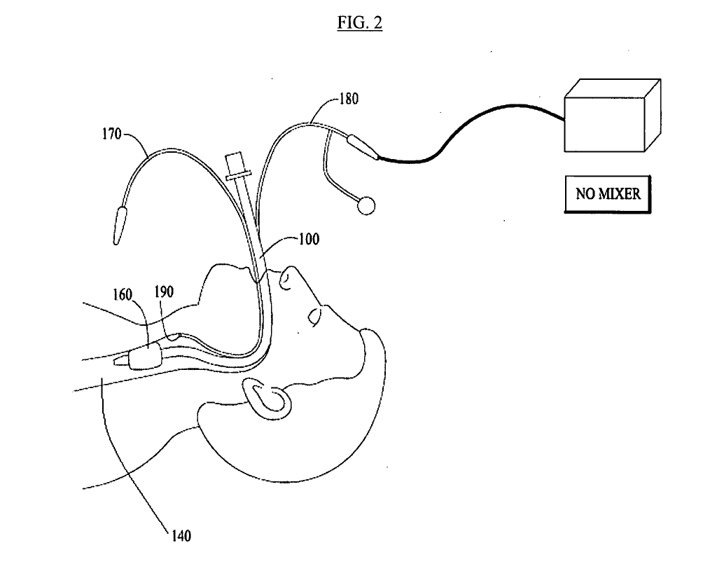

The respiratory assist device can be used and/or modified for use, for example, as an endotracheal tube or as an tracheotomy tube. As shown in FIG. 2, the respiratory assist device can be used as an endotracheal tube by inserting the device 100 into the trachea 140 of the patient though the mouth in order to aid in the mechanical ventilation of the lungs. The inflation balloon cuff 160 seals off the lungs while breathable air is delivered though the central lumen (not illustrated) of the respiratory assist device 100. A nitric oxide gas source connected to a gas mixer, for example, flows nitric oxide gas through the NO gas tube 180 and into the oropharyngeal area of the intubated patients via the exit opening 190 to topically bathe or expose the balloon cuff, the tracheal walls, and any other exposed areas on the surface or subsurface of the oropharyngeal area. Preferably, the concentration of nitric oxide gas delivered to this area ranges from about 100 ppm to about 20,000 ppm, and more preferably from about 160 ppm to about 200 ppm. Even at relatively high concentrations of NO, it is not anticipated that NO will need to be scavenged in order to prevent its escape from the oropharyngeal area of the patient to the ambient atmosphere because the small volume of gas that is required to inundate the oropharyngeal area quickly will be dissipated and diluted after exiting the intubated patient, for example through the patient's mouth.

In another embodiment, the respiratory assist device can be provided with an aspiration system in order to reduce the amount of secretions that may provide the environment for microbial growth. For example, the exit opening 190 in the exemplary respiratory device depicted in FIG. 1 can act both as an exit hole for nitric oxide gas and as an input hole for the aspiration of the secretions. A switch valve that switches the fluid communication of the tube between the nitric oxide gas source and an aspirator may be located upstream. From time to time, the switch valve is switched to the aspirator such that the secretions can be aspirated to reduce the amount of fluids accumulating on and around the balloon cuff.

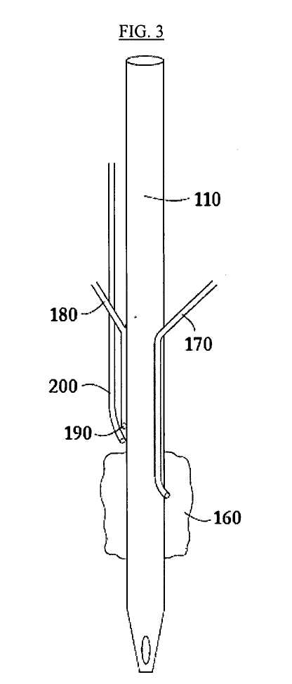

Alternatively, as seen in FIG. 3, the respiratory assist device can include another tube 200 connected to an aspirator, separate from the nitric oxide gas tube 180. Preferably, additional and separate openings in fluid communication only with the aspirating tube 200 are provided on the respiratory assist device such that the flowpath of nitric oxide gas and the flowpath of the aspirate are separate. FIG. 3 also illustrates an alternative configuration with the nitric oxide tube 180 disposed on the exterior of the respiratory assist device and the inflation tube 170 disposed on the interior of the device.

Generally, any respiratory assist tube such as a tracheotomy tube or endotracheal tube can be constructed with tubing in fluid communication with a nitric oxide source to deliver nitric oxide gas to the oropharyngeal area in a patient implanted or receiving a respiratory assist tube. For example, in the case of the tracheotomy tube, exit openings above an inflatable balloon cuff in fluid communication with a source of nitric oxide gas can be provided. In this embodiment, the nitric oxide source preferably is a small canister with pressurized nitric oxide gas that may be easily transportable or carried, but other ways of providing nitric oxide gas as already discussed also can be used.

The devices described herein can be used to practice methods of decontaminating secretions in intubated mammals, and particularly of decontaminating the oropharyngeal area of intubated mammals. The devices also may be used to prevent ventilator acquired pneumonia caused by secretions in intubated mammals and in methods of mechanically ventilating a mammal without causing ventilator acquired pneumonia.

For example, in a method of decontaminating secretions in an intubated mammal, nitric oxide gas is delivered to the secretions in a concentration sufficient to decontaminate the secretions. In a method of decontaminating the oropharyngeal area in an intubated mammal in particular, the oropharyngeal area is sealed from the lungs and an effective concentration of nitric oxide gas is delivered to the sealed area of the oropharyngeal. In a method of mechanically ventilating a mammal without causing ventilator acquired pneumonia, the mammal's trachea is intubated and its lungs mechanically ventilated. An area of the oropharyngeal is sealed from the lungs so that secretions collect in the sealed area and a concentration of nitric oxide gas sufficient to substantially decontaminate the collected secretions is delivered to the sealed area. More particularly, the mammal's trachea can be intubated with a respiratory assist device as described herein and the mammal's lungs ventilated through the catheter of the respiratory assist device. The balloon cuff of the respiratory assist device can be inflated in order to seal an area of the oropharyngeal from the lungs so that secretions collect in the sealed area.

In these exemplary methods, the concentration of nitric oxide gas preferably is from about 100 ppm to about 20,000 ppm, and more preferably from about 160 ppm to about 200 ppm. Additionally, the secretions and/or the sealed oropharyngeal area can be aspirated in order to further the purposes of the methods.

To study the effects of gaseous nitric oxide on potential pathogens, a custom gas exposure incubator was designed and validated for temperature, humidity, and gas concentrations, providing an environment that matches that of a microbiologic incubator, while enabling controlled exposure of precise concentrations of the gas.

For the initial pilot studies, two strains of bacterial pathogen were selected based on two proposed clinical applications of gNO for respiratory infections and topical application. P. aeruginosa, that is associated primarily with pulmonary disease and S. aureus, that is associated with surface wound infections, were chosen for study.

P. aeruginosa is a problematic pathogen that is difficult to treat because of its resistance to antibiotics. It is often acquired in the hospital and causes severe respiratory tract infections. P. aeruginosa is also associated with high mortality in patients with cystic fibrosis, severe burns, and in AIDS patients who are immunosuppressed. Speert, D. P., Molecular Epidemiology of Pseudomonas Aeruginosa, Frontier in Bioscience 7: e354-361 (2002). The clinical problems associated with this pathogen are many, as it is notorious for its resistance to antibiotics due to the permeability barrier afforded by its outer membrane lipopolysaccharide (LPS). The tendency of P. aeruginosa to colonize surfaces in a biofilm phenotype makes the cells impervious to therapeutic concentrations of antibiotics.

S. aureus was selected as the wound microorganism in this study because Staphylococci are known to be significant pathogens that cause severe infections in humans, including endocarditis, pneumonia, sepsis and toxic shock. Methicillin resistant S. aureus (MRSA) is now one of the most common causes of nosocomial infections worldwide, causing up to 89.5% of all staphylococci infection. Narezkina, A., et al., Prevalence of Methicillin-resistant Staphylococcus aureus in different regions of Russia: results of multicenter study, 12th European Congress of Clinical Microbiology and Infectious Diseases (ECMID) #P481 (2002); Milind, K. and Deirbhile, K. Antimicrobial therapy of methicillin resistant Staphylococcus aureus infection, Expert Opin. Pharmacother. 4(2):165-177 (2003). Community outbreaks of MRSA have also become increasingly frequent. Rosenberg, J., Methicillin resistant Staphylococcus aureus (MRSA) in the community. Who's watching?, Lancet 346:132-133 (1995). The main treatment for these infections is the administration of glycopeptides (Vancomycin and Teicoplanin). MRSA have been reported for two decades, but emergence of glycopeptide-resistance in S. aureus—namely glycopeptide intermediate (GISA)—has been reported only since 1997. Hiramatsu, K., Vancomycin resistant Staphylococcus aureus. WHO report of diseases outbreak, (available at www.who.imt/disease-outbreak-news/n1997/june). The glycopeptides are given only parenterally and have many toxic side effects. Hamilton-Miller, J. M., Vancomycin resistant Staphylococcus aureus. A real and present danger?, Infection 30:118-124 (2002). The recent isolation of the first clinical Vancomycin-resistant strains (VRSA) from a patient in USA has heightened the importance and urgency of developing new agents. Bartley, J., First case of VRSA identified in Michigan, Infect. Control Hosp. Epidemiol. 23:480 (2002).

The first step in the process of evaluating the direct effect of gNO on bacteria was to design a simple study to determine what dose, if any, would be an approximate lethal concentration level for microbes. Once an optimal dose was estimated, then a timing study was conducted. For these initial studies, highly dense inoculums of P. aeruginosa and S. aureus suspensions (10⁸ cfu/ml) were plated onto agar plates. These plates were then exposed to various concentrations of gNO in the exposure device in order to evaluate the effect on colony growth.

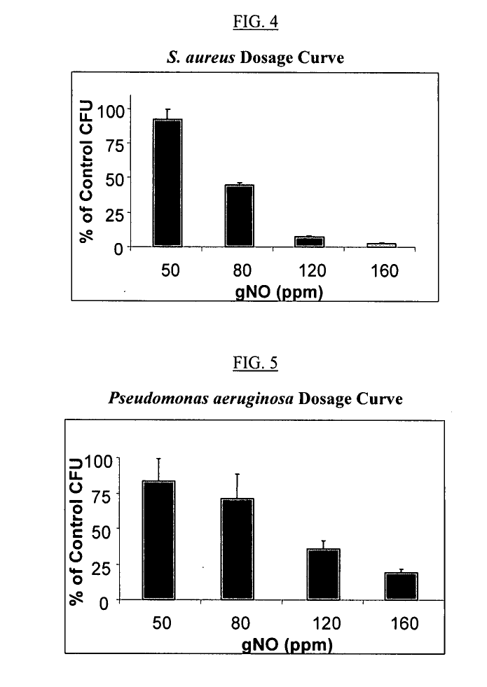

FIGS. 4 and 5 demonstrate that levels of gNO greater than 120 ppm reduced the colony formation of the bacteria by greater than 90%. Further studies indicated that the time required to achieve this affect occurred between 8-12 hours. These results confirm that gNO has an inhibitory effect on P. aeruginosa and S. aureus growth. Additionally, the data provide preliminary evidence that there is a time and dose relationship trend, with the amount of bacteriocidal activity increasing with increased time of exposure and concentration of gNO. As the concentration of gNO increases, the number of colonies growing on the plates decreases.

Although there was a downward bacteriocidal trend towards 5-10% survival with increasing gNO to 120 ppm, none of the data showed a 100% bacteriocidal effect. Some bacteria may have survived because the materials and chemicals in the agar may have reacted with the gNO and buffered the effect. Of significance was the observation that bacterial colonies remained the same in size and number after being transferred to a conventional incubator for 24 hours whereas controls increased in number and size to the degree that they could not be counted. This strongly suggested that gNO exposure prevented the growth of the bacteria, and may have killed the bacteria at some point during the gNO exposure. Accordingly, subsequent studies were designed to further study the bacteriocidal effects of gNO.

Following the dose and time ranging studies, a series of experiments were performed to determine the time required to effectively induce a bacteriocidal effect with 200 parts per million of gNO, a concentration just above the dose used in the dose-ranging study, on a representative collection of drug resistant gram-positive and gram-negative strains of bacteria associated with clinical infection. A successful bacteriocidal effect was defined as a decrease in bacteria greater than 3 log₁₀ cfu/ml. Further, C. albicans, methicillin resistant S. aureus (MRSA), a particularly resistant strain of P. aeruginosa from a cystic fibrosis patient, Group B Streptococcus, and M. smegmatis were also included to see if yeast, a multi-drug resistant strain of bacteria, and actinomycetes have a similar response. These drug-resistant bacteria represent a variety of pathogens that contribute to both respiratory and wound infections.

For these experiments, saline was selected as a suspension media because it would not mask the direct effect of gNO as a bacteriocidal, whereas fully supplemented growth medium might introduce external variables (e.g., buffer or react with gNO). Other media might also provide metabolites and replenish nutrients that produce enzymes that protect bacteria from oxidative and nitrosative damage, thereby masking the effect of gNO. Furthermore, it has been suggested that a saline environment more realistically represents the hostile host environment that bacteria typically are exposed to in vivo. In saline, the colonies were static but remained viable. This is similar to the approach of Webert and Jean's use of animal models. Webert, K. E., et al., Effects of inhaled nitric oxide in a rat model of Pseudomonas aeruginosa pneumonia, Crit. Care Med. 28(7):2397-2405 (2000); Jean D., et al., Beneficial effects of nitric oxide inhalation on pulmonary bacterial clearance, Crit. Care Med. 30(2):442-7 (2002).

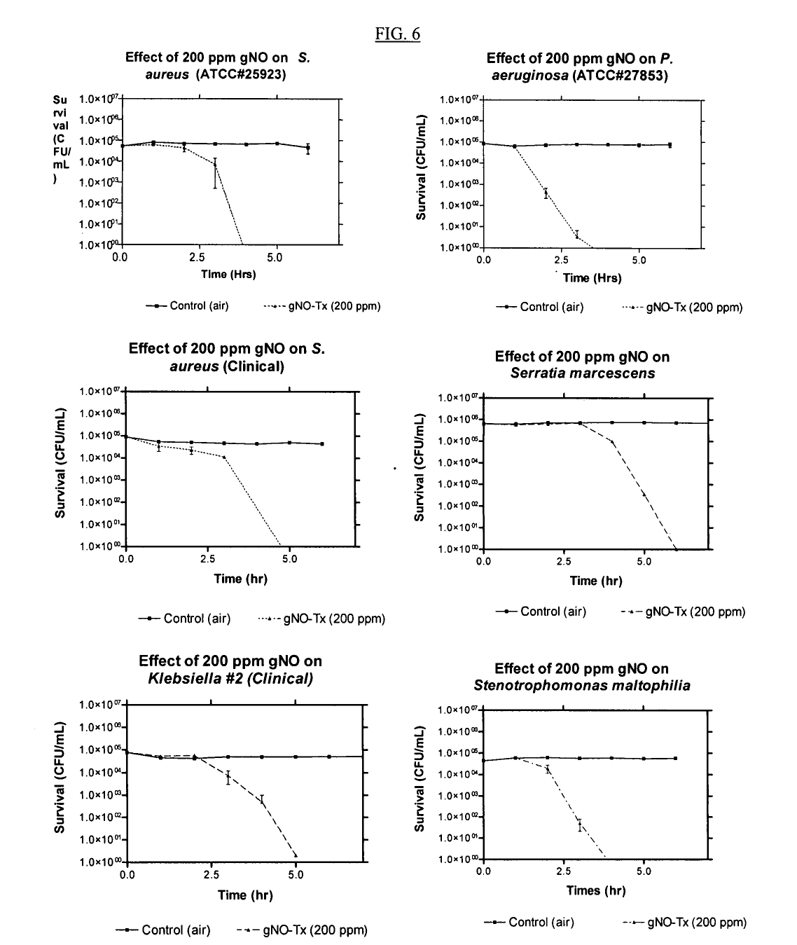

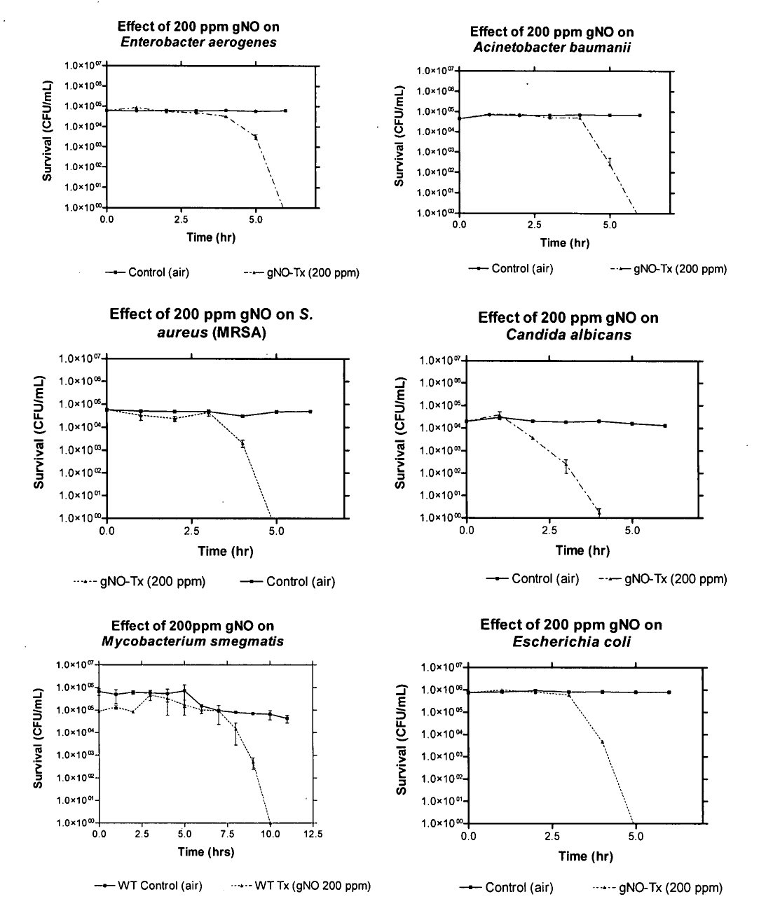

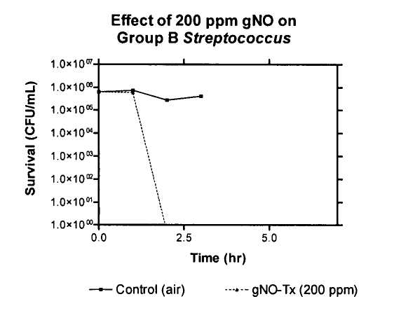

FIG. 6 shows the results of these experiments with survival curves of the control exposure microorganisms plotted against the survival curves of the NO exposed microorganisms. These studies showed that gNO at 200 ppm had a completely bacteriocidal effect on all microorganisms tested. Without exception, every bacteria challenged with 200 ppm gNO had at least a three log₁₀ reduction in cfu/ml and every test resulted in a complete and total cell death of all bacteria. These results were characterized by a period of latency when it appeared that the bacteria were unaffected by gNO exposure (Table 1). The latent period was then followed by an abrupt death of all cells. Gram negative and gram positive bacteria, antibiotic resistant bacterial strains, yeast and mycobacteria were all susceptible to 200 ppm gNO. Of importance is the observation that the two drug resistant bacteria strains were also susceptible.

TABLE 1

| | | Latent | | |
|---|---|---|---|---|
| | Gram | Period | −2.5 Log₁₀ | LD₁₀₀ |
| Bacteria | staining | (hrs) | (hrs) | (Hrs) |
| S. aureus (ATCC) | Positive | 3 | 3.3 | 4 |
| P. aeruginosa | Negative | 1 | 2.1 | 3 |
| (ATCC) | | | | |
| Methicillin resistant | Positive | 3 | 4.2 | 5 |
| S. aureu (MRSA) | | | | |
| Serracia sp. | Negative | 4 | 4.9 | 6 |
| S. aureus (Clinical) | Positive | 3 | 3.7 | 4 |
| Klebsiella sp. #1 | Negative | 3 | 3.5 | 6 |
| Klebsiella sp.#2 | Negative | 2 | 4.1 | 5 |
| Klebsiella sp. #3 | Negative | 3 | 5.1 | 6 |
| S. maltophilia | Negative | 2 | 2.8 | 4 |
| Enterobacter sp. | Negative | 4 | 5.3 | 6 |
| Acinetobacter sp. | Negative | 4 | 5 | 6 |
| E. coli | Negative | 3 | 4.2 | 5 |
| Group B | Positive | 1 | 1.5 | 2 |
| Streptococci | | | | |
| Average | N/A | 2.77 | 3.82 | 4.77 |
| SD | N/A | 1.01 | 1.17 | 1.30 |
| Mycobacterium | Positive | 7 | 9.2 | 10 |
| smegmatis | | | | |

These results show that gNO directly exhibits a non-specific lethal effect on a variety of potentially pathogenic microorganisms. The study also indicates a significant difference in the lag period for mycobacteria compared to all other organisms. The lag period suggests that mycobacteria may have a mechanism that protects the cell from the cytotoxicity of gNO for a longer period than other bacteria.

Applicants believe that there is a dose-time dependent gNO threshold reached within the cell at which point rapid cell death occurs. It is possible that this threshold occurs when the normal NO detoxification pathways of the bacteria are overwhelmed. These studies indicate and confirm that supraphysiologic levels of NO are bacteriocidal on representative strains of drug resistant bacteria. The effect appears to be abrupt, lethal and non-specific on these bacteria.

While embodiments of the present invention have been shown and described, various modifications may be made without departing from the scope of the present invention. The invention, therefore, should not be limited, except to the following claims, and their equivalents.

What is claimed:

1. A method of decontaminating secretions in an intubated mammal, comprising delivering a concentration of nitric oxide gas to the secretions in the intubated mammal sufficient to substantially decontaminate the secretions.

2. The method of claim 1, wherein the mammal is intubated with an endotracheal tube or a tracheotomy tube equipped with a balloon cuff, and secretions collect at the balloon cuff.

3. The method of claim 1, wherein the concentration of nitric oxide gas is from about 100 ppm to about 20,000 ppm.

4. The method of claim 1, wherein the concentration of nitric oxide gas is from about 160 ppm to about 200 ppm.

5. The method of claim 1, further comprising aspirating the secretions from the intubated mammal.

6. A method of decontaminating the oropharyngeal area in an intubated mammal, the method comprising the steps of:
sealing an area of the oropharyngeal from the lungs; and
delivering an effective concentration of nitric oxide gas to the sealed area of the oropharyngeal.

7. The method of claim 6, wherein the mammal is intubated with an endotracheal tube or a tracheotomy tube equipped with a balloon cuff, and wherein sealing an area of the oropharyngeal comprises inflating the balloon cuff.

8. The method of claim 6, wherein the concentration of nitric oxide gas is from about 100 ppm to about 20,000 ppm.

9. The method of claim 6, wherein the concentration of nitric oxide gas is from about 160 ppm to about 200 ppm.

10. The method of claim 6, further comprising aspirating the sealed area of the oropharyngeal.

11. A method of preventing ventilator acquired pneumonia caused by secretions in an intubated mammal, comprising:
sealing an area of the oropharyngeal from the lungs so that secretions collect in the sealed area; and
delivering a concentration of nitric oxide gas to the sealed area sufficient to substantially decontaminate the secretions that collect in the sealed area.

12. The method of claim 11, wherein the mammal is intubated with an endotracheal tube or a tracheotomy tube equipped with a balloon cuff, and wherein sealing an area of the oropharyngeal from the lungs comprises inflating the balloon cuff.

13. The method of claim 11, wherein the concentration of nitric oxide gas is from about 100 ppm to about 20,000 ppm.

14. The method of claim 11, wherein the concentration of nitric oxide gas is from about 160 ppm to about 200 ppm.

15. The method of claim 11, further comprising aspirating the sealed area of the oropharyngeal.

16. A method of mechanically ventilating a mammal without causing ventilator acquired pneumonia, comprising:
intubating the mammal's trachea;
mechanically ventilating the mammal's lungs;
sealing an area of the oropharyngeal from the lungs so that secretions collect in the sealed area; and
delivering a concentration of nitric oxide gas to the sealed area sufficient to substantially decontaminate secretions that collect in the sealed area.

17. The method of claim 16, wherein the mammal is intubated with an endotracheal tube or a tracheotomy tube equipped with a balloon cuff, and wherein sealing an area of the oropharyngeal from the lungs comprises inflating the balloon cuff.

18. The method of claim 16, wherein the concentration of nitric oxide gas is from about 100 ppm to about 20,000 ppm.

19. The method of claim 16, wherein the concentration of nitric oxide gas is from about 160 ppm to about 200 ppm.

20. A method of mechanically ventilating a mammal without causing ventilator acquired pneumonia, comprising:
intubating the mammal's trachea with a catheter having an inflatable balloon cuff positioned at about the distal end of the catheter;
mechanically ventilating the mammal's lungs though the catheter;
inflating the balloon cuff to seal an area of the oropharyngeal from the lungs so that secretions collect in the sealed area; and
delivering a concentration of nitric oxide gas to the sealed area sufficient to substantially decontaminate secretions that collect in the sealed area.

21. The method of claim 20, wherein the concentration of nitric oxide gas is from about 100 ppm to about 20,000 ppm.

22. The method of claim 20, wherein the concentration of nitric oxide gas is from about 160 ppm to about 200 ppm.

23. The method of claim 20, further comprising aspirating the secretions from the intubated mammal.
